(19) United States
(12) Patent Application Publication
(10) Pub. No.: US 2020/0405277 A1
(43) Pub. Date: Dec. 31, 2020

(54) ENDOSCOPIC ULTRASOUND NEEDLE DEVICE AND METHOD FOR OPERATING THE SAME

(71) Applicants: Arthur John Kaffes, Bellevue Hill (AU); Micro-Tech (Nanjing) Co., Ltd., Nanjing (CN)

(72) Inventors: Arthur John Kaffes, Bellevue Hill (AU); Changqing Li, Nanjing (CN); Jianjun Shuang, Nanjing (CN); Gang Zhang, Nanjing (CN); Hongyan Jin, Nanjing (CN); Zhenghua Shen, Nanjing (CN)

(73) Assignees: Micro-Tech (Nanjing) Co., Ltd.; Arthur John Kaffes

(21) Appl. No.: 16/910,970

(22) Filed: Jun. 24, 2020

Related U.S. Application Data
(60) Provisional application No. 62/952,535, filed on Dec. 23, 2019.
(60) Provisional application No. 62/868,392, filed on Jun. 28, 2019.

(51) Int. Cl. A61B 10/04; A61B 10/02; A61B 8/12

(52) U.S. Cl. CPC A61B 10/04; A61B 2010/045; A61B 8/12; A61B 10/0275

(57) ABSTRACT

The present disclosure provides an endoscopic ultrasound needle device and a method for operating the same. The endoscopic ultrasound needle device includes a first needle defining a first bore; a second needle located in the first bore, wherein the second needle is adapted to move or reciprocate within the first bore in response to a manipulation of a component located adjacent or on a handle of the device; and a first cut edge on the first needle, defining an orifice or opening in open communication with the first bore, wherein the first cut edge cooperates with at least a portion of the second needle to cut or slice the tissue, during the second needle moves or reciprocates within the first bore, relative to the first needle, in the extension direction of the first bore, which has high tissue sampling efficiency.

### CROSS-REFERENCE TO RELATED APPLICATIONS

The present disclosure claims priority to U.S. Provisional Patent Application No. 62/868,392, filed with the United States Patent and Trademark Office on Jun. 28, 2019, entitled “Endoscopic Ultrasound Needle”; and

priority to U.S. Provisional Patent Application No. 62/952,535, filed with the United States Patent and Trademark Office on Dec. 23, 2019, entitled “Endoscopic Ultrasound Needle”, which are incorporated herein by reference in its entirety.

### TECHNICAL FIELD

The present disclosure relates to the field of medical apparatus and instruments, and in particular, to an endoscopic ultrasound needle device and a method for operating the same.

### BACKGROUND

Endoscopic ultrasound (EUS) and fine needle aspiration (FNA) is a technique using sound waves during an endoscopic procedure to check up or through the wall of the gastrointestinal tract. Conventionally, a fine needle can be advanced into tissues to obtain an aspirate of the tissue. The cells obtained from the FNA can be applied to a glass slide and analyzed for abnormalities such as cancer. Such cell analysis is called cytology. EUS-FNA has been a great advancement in the ability to diagnose and stage cancers of the gastrointestinal tract and assess the pancreas. Gastrointestinal cancers can be checked up with EUS and their depth of penetration into the intestinal wall can be determined.

Some exemplary EUS-FNA devices exist. For example, the Olympus EZ Shot 2 or 3, the Boston Scientific Expect and Expect Slimline, and the Cook EchoTip ProCore HD are conventional EUS-FNA device.

### SUMMARY

The embodiments of the present disclosure are achieved as follows:

In accordance with one aspect of the present disclosure, the exemplary embodiment may provide a method comprising collecting a certain volume of tissue containing at least three various sample sizes, adapted to be used in glass slides, cell block, and histology, respectively, during only one pass of a distal end of an endoscopic ultrasound needle device through tissue that is to be tested.

In accordance with one aspect, an exemplary embodiment of the present disclosure may provide an endoscopic ultrasound needle device comprising: a first needle defining a first bore; a second needle positioned within the bore, which is adapted to move or reciprocate within the first bore in response to manipulation of a component located adjacent to or on a handle of the device; a first cut edge on the first needle defining an orifice or opening in open communication with the bore, wherein the first cut edge cooperates with at least a portion of the second needle to cut or excise tissue during movement or reciprocation of the second needle within the first bore, relative to the first needle, along an extension direction of the first bore.

In another aspect, an exemplary embodiment of the present disclosure may provide a method for an endoscopic ultrasound needle comprising: inserting a first needle to pass through tissue or medium, wherein the first needle carries a second needle therein; making the second needle move or reciprocate within the first bore defined by the first needle; making a portion of the second needle move or reciprocate, to pass by the first cut edge in the first needle, wherein the first cut edge defines a radially aligned opening in communication with the bore; collecting a sample from excised or cut segments of the tissue or medium in a rapid manner, wherein the sample is of a volume sufficient to be tested in at least one manner.

### BRIEF DESCRIPTION OF DRAWINGS

An exemplary embodiment of the present disclosure is set forth in the following description, is shown in the drawings and is particularly and distinctly pointed out and set forth in the appended claims. The drawings, which are fully incorporated herein and constitute a part of the specification, illustrate various examples, methods, and other exemplary embodiments of various aspects of the present disclosure. It will be appreciated that the element boundaries (e.g., boxes, groups of boxes, or other shapes) shown in the figures represent one example of the boundaries. One of ordinary skill in the art will appreciate that in some examples, one element may be designed as multiple elements or that multiple elements may be designed as one element. In some examples, an element shown as an internal component of another element may be implemented as an external component and vice versa. Furthermore, various elements may not be drawn to scale.

FIG. 1 is a perspective view of an endoscopic ultrasound needle device in accordance with an exemplary aspect for the present disclosure;

FIG. 2 is a perspective view of the endoscopic ultrasound needle of FIG. 1, which shows section of a portion of the handle to reveal components disposed therein;

FIG. 3 is a perspective view of a stylet in accordance with one aspect of the present disclosure;

FIG. 4a is a perspective cross-section view of the distal end of a first needle, wherein the distal end of the first needle is provided therein with a moveable second needle;

FIG. 4b is an operational perspective view of the distal end of the first needle and the second needle, indicating that sharpened edges align to each other to define a collective radial opening, which enables tissue to be collected into an interior bore of the inner needle;

FIG. 5a is a schematic structural view of the first needle and the second needle in the first relative position;

FIG. 5b is a schematic structural view of the first needle and the second needle in the second relative position;

FIG. 5c is a schematic structural view of the first needle and the second needle in a third relative position;

FIG. 6a is an axonometric view of the first needle and the second needle in a diagrammatic operational view of tissue being cut into cut segments for a sample, wherein these cut segments are collected at a fast rate and according to various volume and size requirements for testing;

FIG. 6b is a schematic view of the second needle after sliding toward the distal end relative to the first needle;

FIG. 6c is a schematic view of the second needle after sliding toward the proximal end relative to the first needle;

FIG. 6d is a schematic view of the first needle and the second needle in the first position during the operating process of cutting the tissue into cut segments for a sample;

FIG. 6e is a schematic view of the first needle and the second needle in the second position during the operating process of cutting the tissue into cut segments for a sample;

FIG. 6f is a schematic view of the first needle and the second needle in the third position during the operating process of cutting the tissue into cut segments for a sample;

FIG. 7 is a schematic structural view of a first syringe member provided by the present disclosure;

FIG. 8 is a schematic structural view of a second syringe member provided by the present disclosure;

FIG. 9 is a schematic view of the cooperation between the first lantern ring and the first elongated member provided by the present disclosure;

FIG. 10 is a schematic view of the cooperation between the second lantern ring and the second elongated member provided by the present disclosure;

FIG. 11 is a schematic view of the deformation structure of the endoscopic ultrasonic needle device of the present disclosure.

Similar reference signs refer to similar parts throughout the drawings.

### REFERENCE SIGNS

10—endoscopic ultrasonic needle device; 12—distal end; 14—proximal end; 16—longitudinal axis; 18—stylet; 20—first syringe member; 201—guiding protrusion; 202—limiting protrusion; 22—second syringe member; 221—guiding groove; 222—clamping groove; 2221—abutment groove wall; 24—first elongated member; 25—second elongated member; 26—first tooth; 27—second tooth; 28—third syringe member; 29—outer sheath; 30—first needle; 32—second needle; 34—first lantern ring; 341—first sleeve; 3411—first mounting port; 3412—first limiting bump; 342—first elastic clamping strip; 3421—first pressing section; 3422—first clamping section; 3423—first blocking block; 3424—first clamping block; 343—first arc-shaped groove; 36—elastic member; 38—second lantern ring; 381—second sleeve; 3811—second mounting port; 3812—second limiting bump; 382—second elastic clamping strip; 3821—second pressing section; 3822—second clamping section; 3823—second blocking block; 3824—second clamping block; 383—second arc-shaped groove; 40—first end; 42—second end; 44—elongated main body; 46—opening; 48—first bore; 49—second bore; 50—inner surface; 52—first outer surface; 54—first radial opening; 56—first sharpened edge; 58—second outer surface; 60—distal portion; 62—second radial opening; 64—second sharpened edge; 66—tissue; 68—cut segment; 70—orifice; 72—first cut edge; 721—first matching portion; 722—second matching portion; 74—second cut edge; 741—third matching part; 742—fourth matching part; 743—fifth matching part.

### DETAILED DESCRIPTION OF EMBODIMENTS

The conventional EUS-FNA devices are not without their drawbacks. Accordingly, a need continues to exist for improved EUS-FNA device that can obtain a better or larger sample at one time. The needle-type device of the present disclosure addresses at least one problem of the prior devices by providing an endoscopic ultrasound needle device 10, wherein the endoscopic ultrasound needle device 10 can gather or acquire a certain volume of tissue and three different samples by using once action or movement of the distal end of the needle-type device. The FNA performed by the needle-type device collects the sample from the cut segments that can be placed onto a glass slide. Any core material from the cut segments or sample will be provided to histology and all remaining tissue will be used to make cell blocks. This volume of tissue and three different samples (glass slide, cell block, and histology) will allow for diagnosis at the greatest extent, including histology and genetics.

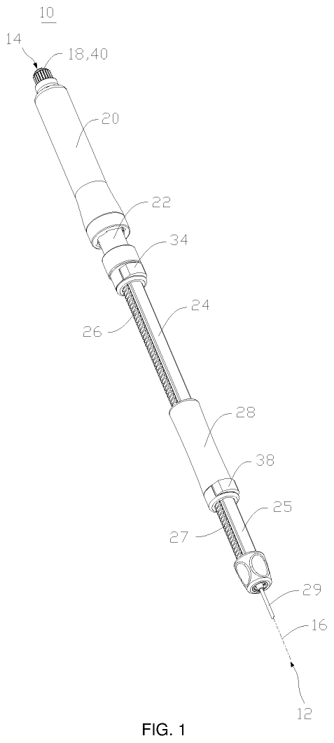

Referring to FIGS. 1 to 11, an exemplary embodiment of the present disclosure may provide an endoscopic ultrasound needle device 10, comprising: a first needle 30 defining a first bore 48; a second needle 32 positioned within the first bore 48, which is adapted to move or reciprocate within the first bore 48, along extension direction of the first bore 48, in response to manipulation of a component adjacent to or on a handle of the device; a first cut edge 72 on the first needle 30 defining an orifice or opening in open communication with the bore, wherein the first cut edge 72 cooperates with at least a portion of the second needle 32 to cut or excise tissue during movement or reciprocation of the second needle 32 within the first bore 48 relative to the first needle along an extension direction of the first bore. Optionally, the tissue may be rapidly collected in a sufficient volume during movement or reciprocation of the second needle 32 along a single axis. Optionally, a second cut edge 74 on the second needle 32 defines an orifice in open communication with the second bore 49 of the second needle 32, wherein the reciprocation of the second needle 32 enables the orifice on the first needle 30 to be aligned with the orifice on the second needle 32 to allow tissue to extend into the first bore 48 through both orifices. Optionally, a distal portion 60 on the second needle 32 is positioned away from the first cut edge 72 on the second needle 32 that defines the orifice, wherein the distal portion 60 on the second needle 32 blocks tissue from entering the first bore 48 through first needle 30 when the distal portion 60 is moved into alignment with the orifice of the first needle 30. Optionally, an outer surface of the second needle 32 is disposed closely adjacent to an inner surface of the first needle 30. In other words, the outer surface of the second needle 32 is fitted to the inner surface of the first needle 30, wherein the outer surface of the second needle 32 refers to the outer surface of the second needle 32 in the circumferential direction, and the inner surface of the first needle 30 refers to the inner surface of the first needle 30 in the circumferential direction. With this arrangement, the first needle 30 can slide more stably relative to the second needle 32 in the extension direction of the longitudinal axis 16.

An exemplary embodiment of the present disclosure may also provide a method for an endoscopic ultrasound needle device, comprising: inserting a first needle 30 to pass through tissue or medium, wherein the first needle 30 carries a second needle 32 therein; making the second needle 32 move or reciprocate within the first bore 48 defined by the first needle 30; making a portion of the second needle 32 move or reciprocate, to pass by the first cut edge 72 in the first needle 30, wherein the first cut edge 72 defines a radially aligned opening in communication with the first bore 48; collecting a sample from excised or cut segments of the tissue or medium in a rapid manner, wherein the sample is of a volume sufficient to be tested in at least one manner. Optionally, the sample is collected in a sufficient volume to be test in three different manners. Optionally, the second needle 32 is moved in a single action to accomplish two times of cut of the tissue or medium.

It should be noted that the first cut edge 72 may be in square, oval, circular, triangular or other shapes.

An endoscopic ultrasound needle in accordance with the present disclosure is shown generally at reference sign 10, and is generally referred to as an endoscopic ultrasound needle device 10, and the endoscopic ultrasound needle device also can be referred to as a needle-type device. The endoscopic ultrasound needle device 10 includes a distal end 12 and a proximal end 14 opposite the distal end 12, wherein a longitudinal axis 16 is defined between the distal end 12 and the proximal end 14. The proximal end 14 is adapted to be grasped by an operator, such as an endoscopic ultrasound inspector, and the distal end 12 is adapted to be in contact with the patient by penetrating patient's tissue.

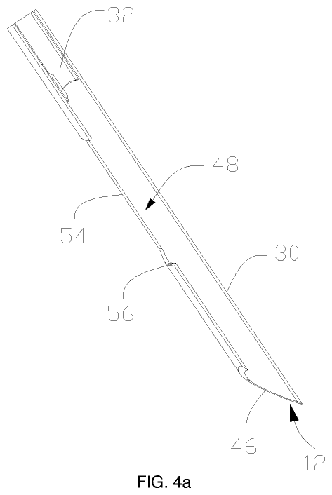

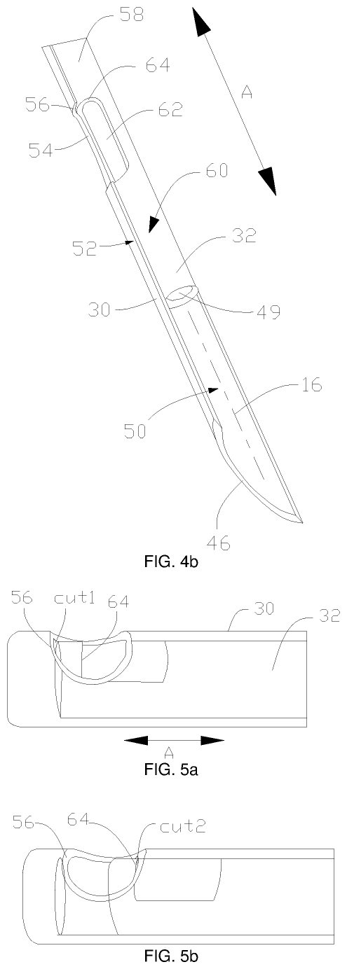

FIG. 1 depicts that the endoscopic ultrasound needle device 10 includes a stylet 18, an external first syringe member 20, an internal second syringe member 22, a first elongated member 24 having multiple first teeth 26 arranged at intervals in the extension direction of the longitudinal axis 16, a third syringe member 28, a first needle 30 (FIG. 4a and FIG. 4b) and a second needle 32 therein (FIG. 4a and FIG. 4b) covered by an outer sheath 29. In addition, there may additionally be a second elongated member 25 having multiple second teeth 27 arranged at intervals in the extension direction of the longitudinal axis 16.

It should be noted that the first syringe member 20, the second syringe member 22, the third syringe member 28, the first elongated member 24 and the second elongated member 25 constitute a handle or a handle assembly.

In the present disclosure, optionally, the stylet 18 is configured to penetrate tissue that has fallen into the second needle 32 after being cut, to facilitate removal of the tissue after being cut.

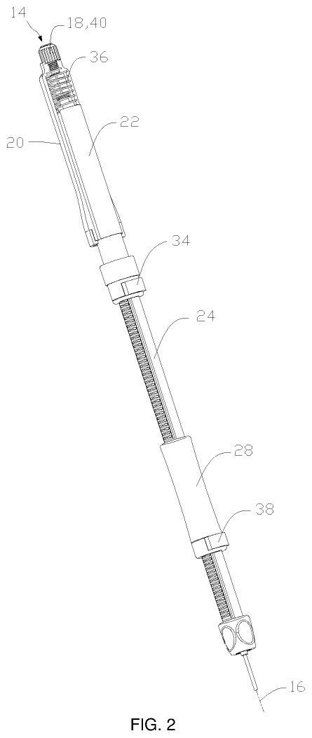

Referring to FIGS. 1 and 2, optionally, the second needle 32 is sleeved outside the stylet 18, the first needle 30 is sleeved outside the second needle 32, and the first needle 30 and the second needle 32 are in sliding fit in the extension direction of the longitudinal axis 16; the outer sheath 29 is sleeved outside the first needle 30, and the first needle 30 and the outer sheath 29 are in sliding fit in the extension direction of the longitudinal axis 16. In the direction from the distal end 12 to the proximal end 14 of the endoscopic ultrasound needle device 10, a second elongated member 25, the third syringe member 28, which is sleeved on the second elongated member 25 and in sliding fit in the extension direction of the longitudinal axis 16, the first elongated member 24 inserted into the proximal end of the third syringe member 28, the second syringe member 22, which is sleeved outside the first elongated member 24 and in sliding fit in the extension direction of the longitudinal axis 16, and the first syringe member 20 sleeved outside the second syringe member 22 are sequentially arranged, the first syringe member 20 and the second syringe member 22 are in sliding fit in the extension direction of the longitudinal axis 16. The outer sheath 29 is connected to the third syringe member 28 to drive the outer sheath 29 to move relative to the second elongated member 25 when the third syringe member 28 slides along the extension direction of the longitudinal axis 16; and the proximal end of the second needle 32 is connected to the proximal end of the first syringe member 20, and the proximal end of the first needle 30 is connected to the proximal end of the second syringe member 22.

It should be noted that, for ease of description, the direction from the proximal end to the distal end in the extension direction of the longitudinal axis 16 is the first direction, and the direction from the distal end to the proximal end is the second direction.

Referring to FIG. 7, optionally, the inner circumferential wall of the first syringe member 20 is provided with a guiding protrusion 201 and a limiting protrusion 202. The guiding protrusion 201 is in a bar shape and extends along the extension direction of the longitudinal axis 16. The number of guiding protrusions 201 is set as needed, and when there are multiple guiding protrusions 201, the multiple guiding protrusions 201 are arranged at intervals in the circumferential direction of the first syringe member 20. In the present disclosure, the number of the guiding protrusions 201 is two and they are arranged symmetrically. The limiting protrusion 202 is located on the side of the distal end of the guiding protrusion 201. The number of the limiting protrusions 202 is set as needed. When the number of the limiting protrusions 202 is multiple, the multiple limiting protrusions 202 are arranged at intervals in the circumferential direction of the first syringe member 20. Optionally, the limiting protrusion 202 has an abutment wall close to the proximal end 14.

Referring to FIG. 8, optionally, the outer circumferential wall of the second syringe member 22 is provided with a guiding groove 221 extending along the extension direction of the longitudinal axis 16 and a clamping groove 222 communicating with the distal end of the guiding groove 221. The clamping groove 222 extends in the circumferential direction of the second syringe member 22 and may be in a ring shape, and the clamping groove 222 has an abutment groove wall close to the distal end 12. The number of the guiding grooves 221 is set as required. When the number of the guiding grooves 221 is multiple, the multiple guiding grooves 221 are arranged at intervals along the circumferential direction of the second syringe member 22. In the present disclosure, the number of the guiding grooves 221 is two and the two guiding grooves 221 are symmetrically arranged.

When the first syringe member 20 is sleeved outside the second syringe member 22, the multiple guiding protrusions 201 and the multiple guiding grooves 221 correspond one-to-one and are in sliding fit, which can play a guiding role when the first syringe member 20 and the second needle member 22 slides relatively in the extension direction of the longitudinal axis 16. Meanwhile, the limiting protrusion 202 is engaged with the clamping groove 222, and the thickness of the limiting protrusion 202 in the extension direction of the longitudinal axis 16 is smaller than the width of the clamping groove 222 in the extension direction of the longitudinal axis 16, thereby ensuring that the limiting protrusion 202 can slide, in the clamping groove 222, in the extension direction of the longitudinal axis 16 relative to the clamping groove 222, thereby ensuring that the first syringe member 20 and the second syringe member 22 can slide relatively. Moreover, when the second syringe member 22 slides in the first direction, the abutment wall can abut against the abutment groove wall, thereby driving the first syringe member 20 to slide together when the second syringe member 22 continues to be driven to slide in the first direction, that is, by operating the second syringe member 22, the first needle 30 and the second needle 32 can be simultaneously driven to move, so as to simultaneously control the depth of insertion of the first needle 30 and the second needle 32 into the patient's tissue. While keeping the second syringe member 22 stationary and operating the first syringe member 20 to move in the first direction, the abutment wall and the abutment groove wall are separated, which will not affect the normal movement of the first syringe member 20, that is, it is achieved that the first syringe member 20 drives the second needle 32 to move in the first direction relative to the first needle 30.

Referring to FIG. 2, optionally, the endoscopic ultrasonic needle device 10 further includes an elastic member 36. The elastic member 36 is disposed between the first syringe member 20 and the second syringe member 22, and is configured so that the first syringe member 20 has a tendency to move in the second direction, so that the abutment wall always remains in contact with the abutment groove wall. When the second syringe member 22 is driven to slide relative to the first elongated member 24 in the first direction, so as to insert the first needle 30 into the patient's tissue, the second needle 32 can be simultaneously driven to be inserted into the patient's tissue.

Optionally, the elastic member 36 may be a spring or other biasing member. In the present disclosure, the elastic member 36 is a spring. The spring is located between the inner surface of the proximal end of the first syringe member 20 and the outer surface of the proximal end of the second syringe member 22. The spring is in a state of compression and has a restoring force for elongation, so that the abutment wall and the abutment groove wall are always in contact with each other. When the second syringe member 22 is kept stationary and the first syringe member 20 is driven to move from the proximal end to the distal end, the spring is further compressed. When the external force applied to the first syringe member 20 is cancelled, under the action of elastic force of the spring, the first syringe member 20 slides relative to the second syringe member from the distal end to the proximal end, and thereby the first syringe member 20 is reset. FIG. 1 and FIG. 2 depict that the first elongated member 24 extends along the longitudinal axis 16 between a first end and a second end. In other words, the first elongated member 24 extends along the longitudinal axis 16 between the proximal end 14 and the distal end 12. The first end of the first elongated member 24 is positioned closely adjacent to the proximal end 14. The first elongated member 24 extends linearly, in length, along the extension direction of the longitudinal axis 16. The second syringe member 22 therein surrounds the first elongated member 24 and may have teeth or grooves that cooperate and interact with the first teeth 26 on the first elongated member 24. The second syringe member 22 has a first end and a second end aligned along the longitudinal axis 16. The first end of the second syringe member 22 is closely adjacent to the first end of the first elongated member 24.

The second end of the second syringe member 22 receives a first lantern ring 34 surrounding the first elongated member 24. The first lantern ring 34 interacts with the second syringe member 22. In other words, the first lantern ring 34 has a locked state and an unlocked state with respect to the first elongated member 24. When the first lantern ring 34 is in the locked state with respect to the first elongated member 24, the first lantern ring 34 and the first elongated member 24 remains relatively fixed in the extension direction of the longitudinal axis 16, that is, the two will not slide relatively in the extension direction of the longitudinal axis 16; when the first lantern ring 34 is in the unlocked state relative to the first elongated member 24, the first lantern ring 34 and the first elongated member 24 are in sliding fit in the extension direction of the longitudinal axis 16, that is, the two can slide relatively in the extension direction of the longitudinal axis 16.

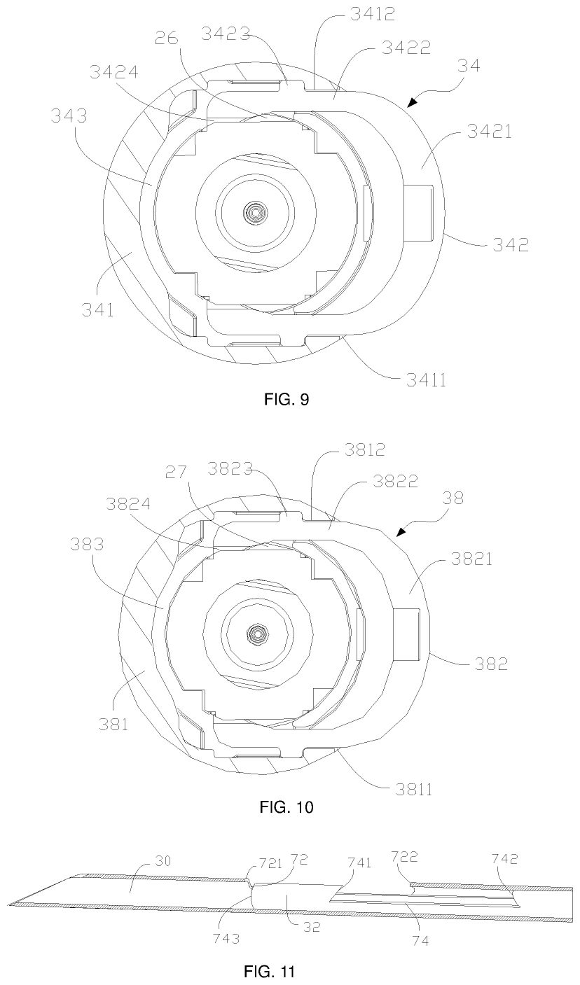

Referring to FIG. 9, optionally, the first lantern ring 34 is connected to the second end of the second syringe member 22, and the two are relatively fixed in the extension direction of the longitudinal axis 16, that is, when the first lantern ring 34 is in the unlocked state relative to the first elongated member 24, when sliding in the extension direction of the longitudinal axis 16 relative to the first elongated member 24, the first lantern ring 34 can drive the second syringe member 22 to slide relative to the first elongated member 24. Meanwhile, when the first lantern ring 34 is in the locked state with respect to the first elongated member 24, the first lantern ring 34 and the first elongated member 24 cannot slide relative to each other in the extension direction of the longitudinal axis 16, that is, the second syringe member 22 cannot slide relative to the first elongated member 24. At this time, the position of the first needle 30 is not easily changed, so that the second needle 32 can be moved relative to the first needle 30 by operating the first syringe member 20.

Optionally, the first lantern ring 34 includes a first sleeve 341 and a first elastic clamping strip 342, wherein the first sleeve 341 is connected to the distal end of the second syringe member 22. The first sleeve 341 is sleeved outside the first elongated member 24, and the first sleeve 341 and the first elongated member 24 are relatively fixed in the circumferential direction of the first elongated member 24. A first mounting port 3411 is provided on the tube wall of the first sleeve 341, and two first limiting bumps 3412 are provided on the inner wall of the first sleeve 341. The two first limiting bumps 3412 are arranged at intervals in the circumferential direction of the first sleeve 341. The first elastic clamping strip 342 includes, in its extension direction, a first pressing section 3421 and two first clamping sections 3422. The first pressing section 3421 is located between the two first clamping sections 3422, and each first clamping section 3422 has a first blocking block 3423 and a first clamping block 3424. The first blocking block 3423 and the first clamping block 3424 are located on opposite sides of the first clamping section 3422. The first elastic clamping strip 342 is inserted into the first mounting port 3411. Under the action of elastic force of the first elastic clamping strip 342 itself, the two first clamping sections 3422 have a tendency to open, so that the two first blocking blocks 3423 abut against the two first limiting bumps 3412, respectively, so as to prevent the first elastic clamping strip 342 from coming out of the first mounting port 3411.

Optionally, the outer circumferential wall of the first elongated member 24 is provided with two groups of first teeth 26, and the two groups of first teeth 26 are arranged oppositely. Each group of first teeth 26 includes multiple first teeth 26, and the multiple first teeth 26 in the same group are arranged at intervals in the extension direction of the longitudinal axis 16, so that the first groove structure is formed between two adjacent first teeth 26.

When the first lantern ring 34 cooperates with the first elongated member 24, the first sleeve 341 is sleeved outside the first elongated member 24, and there is a gap between partial inner circumferential wall of the first sleeve 341 and the outer circumferential surface of the first elongated member 24, which forms a first arc-shaped groove 343 extending along the circumferential direction of the first elongated member 24, and the two first clamping sections 3422 clamp the first elongated member 24, and both the first clamping sections 3422 are located in the first arc-shaped groove 343. The two first clamping blocks 3424 located on the two first clamping sections 3422 and opposite to each other are respectively engaged with the two first groove structures in different groups. There is a gap between the first pressing section 3421 and the outer circumferential wall of the first elongated member 24. In a normal state, the first clamping block 3424 is engaged with the first groove structure formed by two adjacent first teeth 26 in the same group. At this time, the first lantern ring 34 is in a locked state relative to the first elongated member 24. When unlocking is required, the first pressing section 3421 is pressed. Due to the gap between the first pressing section 3421 and the first elongated member 24, the first pressing section 3421 can be close to the first elongated member 24, thereby driving the two first clamping sections 3422 slides in the first arc-shaped groove 343 along the circumferential direction of the first elongated member 24, and the first clamping block 3424 located on the first clamping section 3422 disengages from the first groove structure, and the first clamping block 3424 and the first groove structure are separated from each other, and at this time, the first sleeve 341 can be operated to slide back and forth relative to the first elongated member 24 along the extension direction of the longitudinal axis 16. After sliding to a proper position, the pressure on the first pressing section 3421 is cancelled, and under the action of elastic force of the first elastic clamping strip 342 itself, the first pressing section 3421 is subjected to the restoring deformation, and drives the two first clamping sections 3422 to reset, so that the two first clamping blocks 3424 are engaged with the corresponding first groove structure again to achieve locking.

It should be noted that the first teeth 26 are not limited to two groups, but may be one group. When the first teeth 26 are provided in one group, the first elastic clamping strip 342 is provided with one first clamping block 3424 that cooperates with the first teeth 26. Optionally, the first elastic clamping strip 342 may be a metal strip or a plastic strip.

FIG. 2 depicts that a spring or other biasing member (which is generally referred to as elastic member 36). The elastic member 36 is positioned between the outer surface of the second syringe member 22 at its first end and an inner surface of the external first syringe member 20. The elastic member 36 presses the first syringe member 20 into a neutral first position. The first syringe member 20 is moved by the user against the biasing force of the elastic member 36, so that the first syringe member 20 moves toward the second position in the extension direction of the longitudinal axis 16.

The third syringe member 28 is connected to the distal end of the first elongated member 24. The third syringe member 28 has an inner bore configured for the second elongated member 25 to enter, to adjust the original depth of outer sheath 29 entering the human body. The third syringe member 28 is positioned surrounding a portion of the first elongated member 24. The third syringe member 28 is an independent syringe that is positioned longitudinally from the first lantern ring 34. The third syringe member 28 is retained in a first position along the length of the first elongated member 24 by a second lantern ring 38.

Optionally, the third syringe member 28 is sleeved on the distal end of the first elongated member 24, and the two are relatively fixed in the extension direction of the longitudinal axis; and the third syringe member 28 is sleeved outside the second elongated member 25, the two are connected by a second lantern ring 38, the second sleeve 381 has a locked state and an unlocked state relative to the second elongated member 25, when the second lantern ring 38 is in the locked state relative to the second elongated member 25, the second lantern ring 38 and the second elongated member 25 remain relatively fixed in the extension direction of the longitudinal axis 16, that is, the two will not slide relatively in the extension direction of the longitudinal axis 16; when the second lantern ring 38 is in the unlocked state relative to the second elongated member 25, the second lantern ring 38 and the second elongated member 25 are in sliding fit in the extension direction of the longitudinal axis 16, that is, the two can slide relatively in the extension direction of the longitudinal axis 16. At this time, the second lantern ring 38 drives the third syringe member 28 to slide relative to the second elongated member 25, thereby driving the outer sheath 29 connected to the third syringe member 28 to slide, relative to the second elongated member 25. During the surgery, after the second elongated member 25 is fixed, the outer sheath 29 slides relative to the second elongated member 25, thereby changing the length of the outer sheath 29 extending beyond the second elongated member 25, and finally changing the position, at which the outer sheath 29 is located in the patient's body.

Referring to FIG. 10, optionally, the second lantern ring 38 includes a second sleeve 381 and a second elastic clamping strip 382, wherein the second sleeve 381 is connected to the distal end of the second syringe member. The second sleeve 381 is sleeved outside the second elongated member 25, and the second sleeve 381 and the second elongated member 25 are relatively fixed in the circumferential direction of the second elongated member 25. A second mounting port 3811 is provided on the tube wall of the second sleeve 381, and two second limiting bumps 3812 are provided on the inner wall of the second sleeve 381. The two second limiting bumps 3812 are arranged at intervals in the circumferential direction of the second sleeve 381. The second elastic clamping strip 382 includes, in its extension direction, a second pressing section 3821 and two second clamping sections 3822. The second pressing section 3821 is located between the two second clamping sections 3822, and each second clamping section 3822 has a second blocking block 3823 and a second clamping block 3824. The second blocking block 3823 and the second clamping block 3824 are located on opposite sides of the second clamping section 3822. The second elastic clamping strip 382 is inserted into the second mounting port 3811. Under the action of elastic force of the second elastic clamping strip 382 itself, the two second clamping sections 3822 have a tendency to open, so that the two second blocking blocks 3823 abut against the two second limiting bumps 3812, respectively, so as to prevent the second elastic clamping strip 382 from coming out of the second mounting port 3811.

Optionally, the outer circumferential wall of the second elongated member 25 is provided with two groups of second teeth 27, and the two groups of second teeth 27 are arranged oppositely. Each group of second teeth 27 includes multiple second teeth 27, and the multiple second teeth 27 in the same group are arranged at intervals in the extension direction of the longitudinal axis 16, so that the second groove structure is formed between two adjacent second teeth 27.

When the second lantern ring 38 cooperates with the second elongated member 25, the second sleeve 381 is sleeved outside the second elongated member 25, and there is a gap between partial inner circumferential wall of the second sleeve 381 and the outer circumferential surface of the second elongated member 25, which forms a second arc-shaped groove 383 extending along the circumferential direction of the second elongated member 25, and the two second clamping sections 3822 clamp the second elongated member 25, and both the second clamping sections 3822 are located in the second arc-shaped groove 383. The two second clamping blocks 3824 located on the two second clamping sections 3822 and opposite to each other are respectively engaged with the two second groove structures in different groups. There is a gap between the second pressing section 3821 and the outer circumferential wall of the second elongated member 25. In a normal state, the second clamping block 3824 is engaged with the second groove structure formed by two adjacent second teeth 27 in the same group. At this time, the second lantern ring 38 is in a locked state relative to the second elongated member 25. When unlocking is required, the second pressing section 3821 is pressed. Due to the gap between the second pressing section 3821 and the second elongated member 25, the second pressing section 3821 can be close to the second elongated member 25, thereby driving the two second clamping sections 3822 slides in the second arc-shaped groove 383 along the circumferential direction of the second elongated member 25, and the second clamping block 3824 located on the second clamping section 3822 disengages from the second groove structure, and the second clamping block 3824 and the second groove structure are separated from each other, and at this time, the second sleeve 381 can be operated to slide back and forth relative to the second elongated member along the extension direction of the longitudinal axis 16. After sliding to a proper position, the pressure on the second pressing section 3821 is cancelled, and under the action of elastic force of the second elastic clamping strip 382 itself, the second pressing section 3821 is subjected to the restoring deformation, and drives the two second clamping sections 3822 to reset, so that the two second clamping blocks 3824 are engaged with the corresponding second groove structure again to achieve locking.

It should be noted that the second teeth 27 are not limited to two groups, but may be one group. When the second teeth 27 are provided in one group, the second elastic clamping strip 382 is provided with one second clamping block 3824 that cooperates with the second teeth 27.

In the endoscopic ultrasonic needle device 10 provided by the present disclosure, during the surgery, the second elongated member 25 is fixed to the endoscope, and the third syringe member 28 is driven to slide relative to the second elongated member 25, to adjust the position of the outer sheath 29, at this time, the first elongated member 24, the second syringe member 22 and the first syringe member 20 slide together with the third syringe member 28, correspondingly, the first needle 30 and the second needle 32 slide together relative to the second elongated member 25, after the position adjustment of the third syringe member 28 is completed, the second lantern ring 38 is used to lock the third syringe member 28 relative to the second elongated member 25. At this time, the third syringe member 28 and the second elongated member 25 remain relatively fixed in the extension direction of the longitudinal axis 16. Since the first elongated member 24 is connected to the third syringe member 28, when the position of the third syringe member 28 is locked, the position of the first elongated member 24 is also locked. Then, by driving the second syringe member 22 to slide in the first direction relative to the first elongated member 24, the first syringe member 20 is driven to slide in the first direction relative to the first elongated member 24, so as to achieve that the first needle 30 and the second needle 32 are moved together, that is, the position of the first needle 30 inserted into the patient's tissue is adjusted. After the position adjustment of the second syringe member 22 is completed, the second syringe member 22 is locked relative to the first elongated member 24 by using the first lantern ring 34. Finally, by operating the first syringe member 20 to slide relative to the second syringe member 22 in the first direction, the second needle 32 is driven to slide relative to the first needle 30, so as to achieve cutting, slicing or resection of partial tissue.

The first elongated member 24 is centered about longitudinal axis 16 and defines a bore extending fully therethrough. The bore of the first elongated member 24 extends from its first end to its second end. The bore extending fully through the first elongated member 24 receives the stylet 18, and at least a portion of one of the first needle 30 or the second needle 32 therein. In some instances, a significant portion of both the first needle 30 and the second needle 32 extend through the bore in the first elongated member 24. As described in greater detail herein, the first needle 30 and the second needle 32 are centered concentrically coaxially along longitudinal axis 16. The second needle 32 is configured to move relative to the first needle 30. Optionally, movement of the second needle 32 is accomplished by moving the first syringe member 20 longitudinally along the longitudinal axis 16 exterior to the first elongated member 24. Apparently, by keeping the position of the first needle 30 unchanged, the second needle 32 can be driven to move along the extension direction of the longitudinal axis 16 by operating the first elongated member 24, so as to achieve the relative movement of the first needle 30 and the second needle 32.

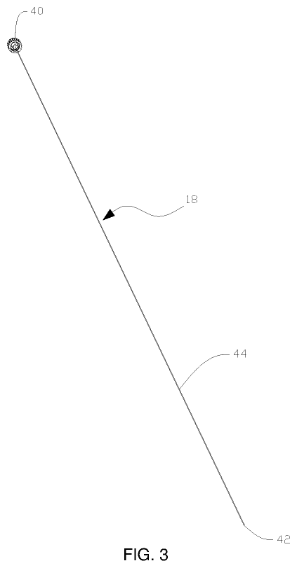

FIG. 3 depicts that stylet 18 has a first end 40, a second end 42, and elongated main body 44 extending between the first end 40 and the second end 42 along the longitudinal axis 16. The elongated main body 44 may have a cross section with any size, as long as the size of the elongated main body 44 can be installed in the bore of the first elongated member 24. Optionally, the elongated main body 44 of the stylet 18 may be disposed within the first needle 30.

FIG. 4a and FIG. 4b depict the first needle 30 and the second needle 32 in a cross-section view. The end of first needle 30 is sharpened and defines the distal end 12 of the endoscopic ultrasonic needle device 10. The sharpened end of the first needle 30 defines an opening 46 that is in open communication with the first bore 48 of the first needle 30. The first bore 48 is centered along the longitudinal axis 16 and is defined by a concave inner surface 50. The concave inner surface 50 is centered along the longitudinal axis 16 and extends circumferentially about the longitudinal axis 16. The first outer surface 52 of the first needle 30 is convexly curved and extends circumferentially about the longitudinal axis 16. Optionally, the radially aligned thickness (relative to axis) is defined by the thickness between inner surface 50 and first outer surface 52.

Optionally, the first needle 30 may be a circular tube; and the second needle 32 may be a circular tube.

Optionally, a first radial opening 54 is defined on the first needle 30 and the first radial opening 54 extends radially through the thickness of the first needle 30 fully between the inner surface 50 and the first outer surface 52. The first radial opening 54 is formed by a first sharpened edge 56. Optionally, the first sharpened edge 56 has a greater length in the longitudinal direction than in the circumferential direction. Thus, the first radial opening 54 is longitudinally elongated defined by the first sharpened edge 56. As described in greater detail herein, the first sharpened edge 56 cooperates with the movement of the second needle 32 to slice, cut, or otherwise extract a tissue sample of a patient in response to operation by the user or operator.

The first radial opening 54 may be in open communication with the first bore 48 of the first needle 30 depending on the position of the second needle 32 disposed within the first bore 48 of the first needle 30. Optionally, the second needle 32 is configured to move longitudinally within the first bore 48 as indicated by Arrow A. The longitudinal movement in the directions of Arrow A (i.e., forward and backward) makes the second outer surface 58 of the second needle 32 aligned closely with the inner surface 50 of the first needle 30. A distal portion 60 of the second outer surface 58 of the second needle 32 can slidably move closely adjacent to the inner surface 50 of the first needle 30. Depending on the position of the second needle 32 within the first bore 48 of the first needle 30, the distal portion 60 of the second outer surface 58 of the second needle 32 may block or occupy the space of the first radial opening 54. The distal portion 60 would preclude or prevent the open communication of the first radial opening 54 with the first bore 48.

Optionally, the second needle 32 defines a second radial opening 62 defined by a second sharpened edge 64, and the second sharpened edge 64 moves past the first sharpened edge 56 during longitudinal translation of the second needle 32 as indicated by Arrow A. Optionally, the second sharpened edge 64 may have similar dimension and shape with the first sharpened edge 56 of the first needle 30. However, it is possible for the second sharpened edge 64 of the second needle 32 to have a different shape than that of the first sharpened edge 56.

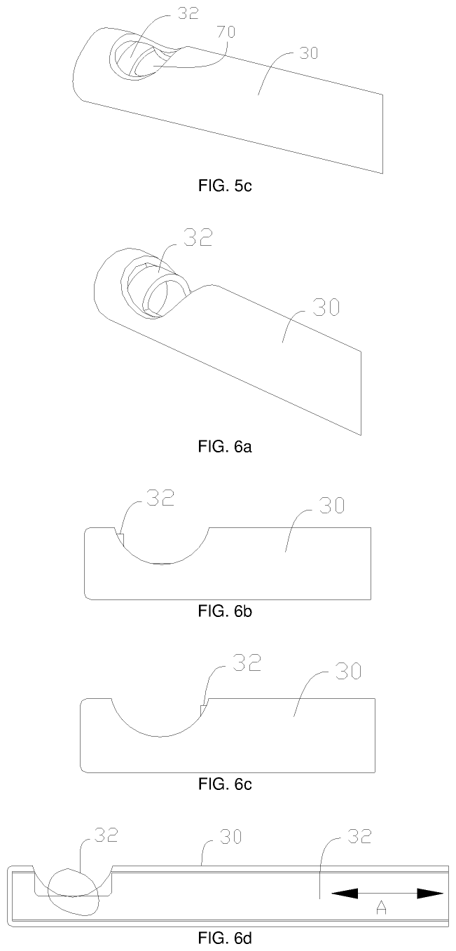

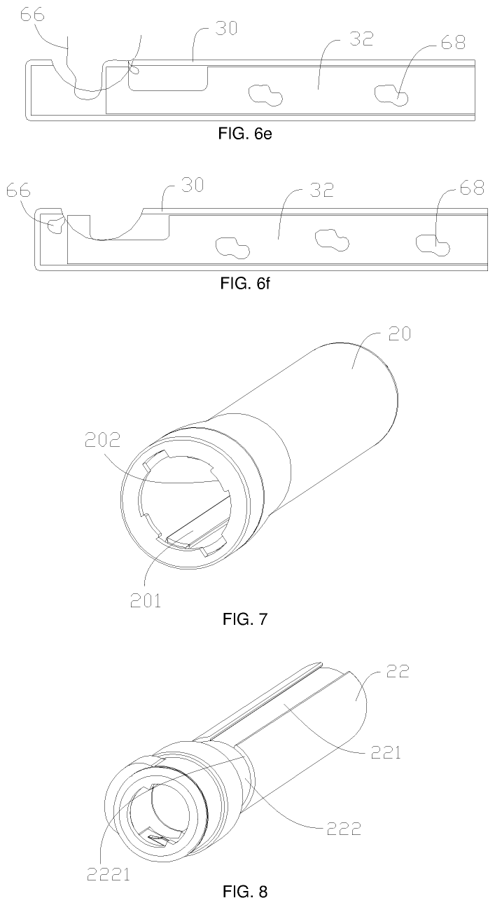

Referring to FIG. 5a, FIG. 5b, FIG. 5c and FIG. 6, the first sharpened edge 56 on the first needle 30 and the second sharpened edge 64 on the second needle 32 create two times of cut with once movement of the second needle 32. Stated otherwise, there is a dual or twin duty cycle to cut tissue based on the single movement of the second needle 32. This is distinguishable from conventional endoscopic ultrasound needles that need multiple movements to excise a tissue sample. Thus, the dual cutting action generated by a single movement of the second needle 32 enables the first sharpened edge 56 and the second sharpened edge 64 to cooperate to cut tissue extending through the aligned second radial opening 62 and first radial opening 54 as the second needle 32 moves longitudinally in the directions of Arrow A, in the distal and proximal direction.

FIG. 5c further depicts the cutting action of the second needle 32 moving distally in the directions of Arrow A along the extension direction of the longitudinal axis 16, such that a first cut is made during the distal movement of the second needle 32 by the interaction of the first sharpened edge 56 with the second sharpened edge 64. A second cut is made during the retraction of the second needle 32 towards the proximal end 14 of the endoscopic ultrasonic needle device 10, to create a second cut based on the interaction of the first sharpened edge 56 with the second sharpened end 64. Thus, the single cutting action referred to herein can be suitable for the movement along one axis back and forth to complete one cycle, and the single cutting action may also be called a single action, a single operation, or a single cutting operation.

FIGS. 6a-6f depict distal and proximal movement of the second needle 32 relative to the first needle 30 as indicated by Arrow A. The tissue 66 may be moved through the orifice 70 formed by the radially aligned first radial opening 54 and second radial opening 62 when the first radial opening 54 and the second radial opening 62 are aligned to each other. The tissue 66 may be moved through the radially aligned first radial opening 54 and second radial opening 62 into the second bore 49. The movement of the second needle 32, as indicated by Arrow A, will slice or cut the tissue 66 to create cut segments 68 of the tissue, which are captured within the second bore 49 inside the second needle 32. The cut segments 68 can be cut from tissue 66 provided that the tissue 66 extends through the radially aligned first radial opening 54 and second radial opening 62 regardless of the longitudinal direction in which the second needle 32 is moving relative to the first needle 30.

As indicated previously, the movement of the second needle 32 is accomplished by moving the first syringe member 20 relative to the second syringe member 22. The movement of the second needle 32 is thus in operable communication with at least one of the first syringe member 20 and the second syringe member 22, such that the biasing force applied by the elastic member 36 can assist in one-direction movement of the second needle 32. For example, an operator may move the first syringe member 20 longitudinally along the longitudinal axis 16, which in turn imparts a translating force, so as to move the second needle 32 from the proximal end to the distal end to cut the tissue 66 and create the cut segments 68 of tissue 66 (i.e., by the first cut). Then, the elastic member 36 may press the first syringe member 20 or second syringe member 22 back to their neutral or resting position, so as to retract the second needle 32 from the distal end to the proximal end, so as to create a second cut for tissue 66 to create the cut segments 68 (i.e., by the second cut) during the retraction or movement of the second needle 32 in the proximal direction.

In the present disclosure, optionally, one of the first cut edge 72 surrounding the first radial opening 54 and the second cut edge 74 surrounding the second radial opening 62 may be provided as a sharpened edge, which can also achieve that when the first needle 30 and the second needle 32 slide along the extension direction of the longitudinal axis 16, the tissue 66 passing through the orifice 70 is cut to obtain the cut segments 68.

It should be understood that at least one of the first cut edge 72 and the second cut edge 74 may be provided as an annular edge. That is, the first cut edge 72 may be an annular edge; or, the second cut edge 74 may be an annular edge; or, both the first cut edge 72 and the second cut edge 74 may be provided as annular edges.

It should be noted that the first cut edge 72 and the second cut edge 74 may be in square, oval, circular, triangular or other shapes.

Referring to FIG. 11, in the present disclosure, optionally, the first cut edge 72 surrounding the first radial opening 54 has a first matching portion 721 and a second matching portion 722 in the extension direction of the longitudinal axis 16, wherein the first matching portion 721 is close to the distal end 12, and the second matching portion 722 is close to the proximal end 14; the second cut edge 74 surrounding the second radial opening 62 has a third matching portion 741 and a fourth matching portion 742 in the extension direction of the longitudinal axis 16, wherein the third matching portion 741 is close to the distal end 12, and the fourth matching portion 742 is close to the proximal end 14. The first matching portion 721 and the fourth matching portion 742 work together to cut the tissue 66 passing through the orifice 70, wherein at least one of the first matching portion 721 and the fourth matching portion 742 is set as a cutting edge, in other words, one of the first matching portion 721 and the fourth matching portion 742 may be set as a cutting edge, or both the first matching portion 721 and the fourth matching portion 742 are set as the cutting edges, both of which are capable of cutting the tissue 66 located in the orifice 70 when relatively sliding along the extension direction of the longitudinal axis 16; and the second matching portion 722 and the third matching portion 741 work together to cut the tissue 66 passing through the orifice 70, wherein at least one of the second matching portion 722 and the third matching portion 741 is set as a cutting edge, in other words, one of the second matching portion 722 and the third matching portion 741 may be set as a cutting edge, or both the second matching portion 722 and the third matching portion 741 are set as the cutting edges, both of which are capable of cutting the tissue 66 located in the orifice 70 when relatively sliding along the extension direction of the longitudinal axis 16.

In the present disclosure, an initial state is set that the second radial opening 62 is closer to the proximal end of the endoscopic ultrasound needle device than the first radial opening 54, in other words, the distal portion 60 blocks the first radial opening 54. At this time, by operating the first syringe member 20 to drive the second needle 32 to slide firstly in the first direction relative to the first needle 30, the first radial opening 54 and the second radial opening 62 are aligned to form an orifice 70, and the tissue 66 extends into the orifice 70. The second needle 32 continues to be driven in the first direction, the fourth matching portion 742 gradually approaches the first matching portion 721, and the first cut is completed under the joint action of the two; then, under the action of the elastic force of the elastic member, the second needle 32 moves relative to the first needle 30 in a second direction opposite to the first direction, and the overlapping portion of the first radial opening 54 and the second radial opening 62 gradually increases, and until the two are aligned in the radial direction to form the orifice 70, the portion of the tissue 66 extends in the radial direction into the orifice 70, and the second needle 32 continues to move in the second direction under the action of the elastic force, the third matching portion 741 gradually approaches the second matching portion 722, and the second cut is achieved under the corporation of the two, that is, a single cutting action can realize the sampling of the tissue 66 twice.

It should be noted that the third matching portion 741 and the fourth matching portion 742 may be set as a barbed structure.

In the present disclosure, optionally, the distal end of the second needle 32 is provided with a fifth matching portion 743, which works together with the first matching portion 721 to cut tissue 66 through the first radial opening 54, wherein at least one of the fifth matching portion 743 and the fourth matching portion 742 is set as a cutting edge. In the initial state, the second radial opening 62 is closer to the proximal end of the endoscopic ultrasound needle device than the first radial opening 54, and the distal portion 60 does not block the first radial opening 54, and the tissue 66 extends in the radial direction into the first radial opening 54. By operating the first syringe member 20 to drive the second needle 32 to slide firstly in the first direction relative to the first needle 30, the fifth matching portion 743 and the first matching portion 721 work together to enable the tissue located in the first radial opening 54 to be cut, for achieving the first cut. Then, the second needle 32 continues to move in the first direction, the first radial opening 54 and the second radial opening 62 are aligned to form the orifice 70, the tissue 66 extends into the orifice 70, and the fourth matching portion 742 gradually approaches the first matching portion 721, and the second cut is completed under the joint action of the two; then, under the action of the elastic force of the elastic member, the second needle 32 moves in the second direction opposite to the first direction relative to the first needle 30, the overlapping portion of the first radial opening 54 and the second radial opening 62 gradually increases, and until the two are aligned in the radial direction to form the orifice 70, the portion of the tissue 66 extends in the radial direction into the orifice 70, and under the action of the elastic force, the second needle 32 continues to move in the second direction, the third matching portion 741 gradually approaches the second matching portion 722, and the third cut is achieved under the cooperation of the two, and at this time, the second needle 32 returns to the initial state, which facilitates the next sampling of the tissue 66, that is, a single cutting action can achieve three times of sampling the tissue 66.

In the present disclosure, optionally, the first cut edge 72 includes a first matching portion 721 and a second matching portion 722, the second cut edge 74 includes a third matching portion 741 and a fifth matching portion 743, and the fifth matching portion 743 and the first matching portion 721 work together to achieve the first cut for the tissue; and the third matching portion 741 and the second matching portion 722 work together to achieve the second cut, thereby achieving two times of cut during a single cutting action.

It should be noted that the cutting edge may be a sharpened edge.

In the present disclosure, the portion of the first cut edge 72 is provided as a sharpened edge, and the portion of the second cut edge 74 is provided as a sharpened edge, thereby reducing processing difficulty, improving processing efficiency, and saving processing cost.

It should be noted that the first cut edge 72 may be configured as a cutting edge as a whole, that is, the first matching portion 721 and the second matching portion 722 are integrated. The second cut edge 74 may be configured as a cutting edge as a whole, that is, the third matching portion 741 and the fourth matching portion 742 are integrated.

In the endoscopic ultrasonic needle device provided by the present disclosure, during the movement of the second needle 32 relative to the first needle 30 along the extension direction of the first bore 48 in the first bore 48 for a single cutting action, the first cut edge 72 cooperates with at least a portion of the second needle 32 to achieve at least two times of cut or excision for tissue extending into the area enclosed by the first cut edge 72. In the process of the single cutting action, the two paired matching portions configured to realize tissue cutting will not be paired repeatedly for multiple times of cut. In the present disclosure, optionally, the endoscopic ultrasonic needle device 10 may further include, at its distal end 12, an endoscope or other viewing device to enable the endoscopic ultrasonic needle device 10 to be pushed to a lesion under real time guidance. The EUS-FNA and core biopsies are performed after Doppler assessment to avoid puncturing intervening blood vessels. Once an organ wall or other bodily wall is punctured by the distal end 12 of the endoscopic ultrasonic needle device 10, the stylet 18 is withdrawn. The distal end 12 of the endoscopic ultrasonic needle device 10 is then moved back and forth through the lesion until the physician or other operator deems it adequate. During the needle moves back and forth through the lesion, the second needle 32 may move longitudinally relative to the first needle 30. The cut segments 68 collected during the operation of the endoscopic ultrasonic needle device 10 define a sample that may be tested in accordance with institutional protocols. The cut segments 68 are stored in the interior of the endoscopic ultrasonic needle device 10 when the endoscopic ultrasonic needle device 10 is removed from the patient. The cut segments 68 defining the sample may then be removed from the endoscopic ultrasonic needle device 10 and tested under traditional protocols.

The endoscopic ultrasonic needle device 10 of the present disclosure may be used in conjunction with a suction device (not shown in figures) that is in operable communication with the second needle 32 and the first needle 30. Namely, the suction device may create a vacuum so as to pull the tissue 66 through the radially aligned first radial opening 54 and second radial opening 62 when the distal end 12 of the endoscopic ultrasonic needle device 10 is inserted into a tissue to be sampled. The suction device creates a vacuum, or otherwise draws the tissue through the radially aligned openings, and the linear movement along the longitudinal axis 16 of the second needle 32 relative to the first needle 30 causes the tissue 66 to be cut while the tissue is drawn through the radially aligned first radial opening 54 and second radial opening 62 based on the suction imparted to the tissue. By relying on the suction device, the endoscopic ultrasonic needle device 10 is able to gather tissue samples from a tumor or other tissue 66 without having to move the needle back and forth to different positions therein. Optionally, prior art needles, when in use, require that the operator or surgeon inserts these types of needles into the tumor or tissue in multiple positions to gather appropriate tissue samples, and such conventional movement of prior art needles causes the needle to cut the tumor or tissue and gather a core sample. In contradistinction to the conventional teachings, the endoscopic ultrasonic needle device 10 can be inserted into a place within the tumor or tissue 66 and the suction can be used to pull the tissue 66 through the radially aligned first radial opening 54 and second radial opening 62 and cut the tissue 66 to gather a better core sample, which enables endoscopic ultrasonic needle device 10 to gather a more sufficient core sample without having to move the device to different positions within the tissue or tumor.

Various inventive concepts may be embodied as one or more methods, of which an example has been provided. The actions performed as part of the method may be ordered in any suitable way. Accordingly, embodiments may be constructed in which actions are performed in an order different than illustrated, which may include performing some actions simultaneously, even though shown as sequential actions in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which teachings of the present disclosure are used. Those skilled in the art will recognize, or be able to ascertain using no more than routine experimentation, many equivalent solutions to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements), among other situations. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”. “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that other elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements), among other situations.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may also be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent to” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the drawings. For example, if a device in the drawings is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only, unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present disclosure.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the present disclosure. The various appearances “an embodiment,” “one embodiment,” “some embodiments,” “one particular embodiment,” or “other embodiments,” or the like, are not necessarily all referring to the same embodiment.

If this specification states a component, feature, structure, or characteristic “may”, “might”, or “could” be included, that particular component, feature, structure, or characteristic is not required to be included. If the specification or claims refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, any method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

In the foregoing description, certain terms have been used for brevity, clarity, and understanding. No unnecessary limitations are to be implied therefrom beyond the requirement of the prior art because such terms are used for descriptive purposes and are intended to be broadly construed.

Moreover, the description and illustration of various embodiments of the present disclosure are examples and the present disclosure is not limited to the exact details shown or described.

### INDUSTRIAL APPLICABILITY

In summary, the present disclosure provides an endoscopic ultrasound needle device and a method for operating the same, which have high tissue sampling efficiency.

1. An endoscopic ultrasound needle device, comprising
a first needle, provided with a first bore;
a second needle, positioned within the first bore, wherein the second needle moves or reciprocates within the first bore in response to a manipulation of a component located adjacent to or on a handle of the device; and
a first cut edge, on the first needle, wherein the first cut edge defines an orifice or opening in communication with the first bore, wherein the first cut edge cooperates with at least a portion of the second needle to cut or excise a tissue during the second needle is moving or reciprocating within the first bore, relative to the first needle, in an extension direction of the first bore.

2. The endoscopic ultrasound needle device according to claim 1, wherein during the second needle is moving within the first bore relative to the first needle in the extension direction of the first bore to perform a single cutting action, the first cut edge cooperates with at least a portion of the second needle to cut or excise, for at least two times, a tissue extending into an area enclosed by the first cut edge.

3. The endoscopic ultrasound needle device according to claim 1, wherein a tissue of a sufficient volume is able to be collected rapidly during the second needle is moving or reciprocating along a single axis.

4. The endoscopic ultrasound needle device according to claim 2, further comprising:
a second cut edge on the second needle, wherein the second cut edge is provided with an orifice in communication with the second bore of the second needle,
wherein a reciprocation of the second needle enables the orifice on the first needle to be aligned with the orifice on the second needle, to allow the tissue to extend into bores through two orifices simultaneously.

5. The endoscopic ultrasound needle device according to claim 4,
wherein the first cut edge is set as an annular sharpened edge.

6. The endoscopic ultrasound needle device according to claim 4,
wherein the second cut edge is set as an annular sharpened edge.

7. The endoscopic ultrasound needle device according to claim 4,
wherein the first cut edge comprises a first matching portion and a second matching portion arranged at intervals in the extension direction of the first bore, and the first matching portion is closer to a distal end of the endoscopic ultrasound needle device than the second matching portion; the second cut edge comprises a third matching portion and a fourth matching portion arranged at intervals in the extension direction of the second bore, and the third matching portion is closer to the distal end of the endoscopic ultrasound needle device than the fourth matching portion; and during the second needle is moving or reciprocating within the first bore relative to the first needle in the extension direction of the first bore, the first matching portion and the fourth matching portion work together to cut a tissue located in the second bore, and the second matching portion and the third matching portion work together to cut the tissue located in the second bore.

8. The endoscopic ultrasound needle device according to claim 7,
wherein at least one of the first matching portion and the fourth matching portion is set as a cutting edge.

9. The endoscopic ultrasound needle device according to claim 7,
wherein at least one of the second matching portion and the third matching portion is set as a cutting edge.

10. The endoscopic ultrasound needle device according to claim 7,
wherein a fifth matching portion is provided at a distal end of the second needle,
wherein during the second needle is moving or reciprocating within the first bore relative to the first needle in the extension direction of the first bore, the fifth matching portion and the first matching portion work together to cut a tissue located in the first bore.

11. The endoscopic ultrasound needle device according to claim 10,
wherein at least one of the fifth matching portion and the first matching portion is set as a cutting edge.

12. The endoscopic ultrasound needle device according to claim 4, further comprising:
a distal portion on the second needle, positioned away from an edge on the second needle defining the orifice,
wherein the distal portion on the second needle blocks the tissue from entering the first bore through the first needle when the distal portion is moving into alignment with the orifice of the first needle.

13. The endoscopic ultrasound needle device according to claim 1, further comprising:
an outer surface of the second needle, disposed closely adjacent to an inner surface of the first needle.

14. The endoscopic ultrasound needle device according to claim 1, further comprising:
an outer surface of the second needle, attached to an inner surface of the first needle.

15. The endoscopic ultrasound needle device according to claim 1, further comprising:
an outer sheath covering the first needle and the second needle, wherein the outer sheath is adapted to avoid damage to a channel of an endoscopic by a tip of the first needle.

16. The endoscopic ultrasound needle device according to claim 1,
wherein the handle comprises a first syringe member and a second syringe member, wherein the first syringe member and the second syringe member are in sliding fit, and the first needle is connected to the second syringe member, and the second needle is connected to the first syringe member.

17. The endoscopic ultrasound needle device according to claim 16,
wherein the handle further comprises an elastic member, wherein the elastic member is simultaneously connected to the first syringe member and the second syringe member, and is configured to cause the first syringe member to trends to move away from the second syringe member.

18. A method for an endoscopic ultrasound needle device, comprising:
inserting a first needle to pass through a tissue or medium, wherein the first needle is provided therein with a second needle;
making the second needle move or reciprocate within a first bore defined by the first needle;
making a portion of the second needle move or reciprocate, to pass by an first cut edge on the first needle, wherein the first cut edge defining a radially aligned opening in communication with the first bore; and
collecting a sample from excised or cut segments of the tissue or medium in a rapid manner, wherein the sample is of a volume sufficient to be tested in at least one manner.

19. The method according to claim 18, wherein a collected sample is in a volume sufficient to be tested in three different manners.

20. The method according to claim 18, further comprising:
the tissue or medium cutting for two times, during moving the second needle in a single action.
